(19) United States
(12) Patent Application Publication
(10) Pub. No.: US 2023/0181371 A1
(43) Pub. Date: Jun. 15, 2023

(54) POLYACRYLONITRILE BASED ELECTROSPUN NANOFIBERS LOADED WITH ZINC OXIDE- QUERCETIN NANOPARTICLES FOR WOUND HEALING

(71) Applicants: Muhammad Raza Shah, Karachi (PK); Tooba Jabri, Karachi (PK); Kanwal Muhammad Iqbal, Karachi (PK); Talat Roome, Karachi (PK); Shafi Ullah, Karachi (PK); Muhammad Qamar Khan, Karachi (PK)

(72) Inventors: Muhammad Raza Shah, Karachi (PK); Tooba Jabri, Karachi (PK); Kanwal Muhammad Iqbal, Karachi (PK); Talat Roome, Karachi (PK); Shafi Ullah, Karachi (PK); Muhammad Qamar Khan, Karachi (PK)

(21) Appl. No.: 17/549,943

(22) Filed: Dec. 14, 2021

(51) Int. Cl. A61F 13/00; D04H 1/728; B82Y 30/00; B82Y 40/00; B82Y 5/00

(52) U.S. Cl. CPC A61F 13/00017; A61F 13/00025; D04H 1/728; A61F 2013/00217; B82Y 5/00; B82Y 30/00; B82Y 40/00; D10B 2321/10; D10B 2401/13; D10B 2509/022

(57) ABSTRACT

Chronic wounds are medical care concern and severe clinical challenge worldwide. A nanofiber based new wound dressing scaffold and a method of developing thereof for treating diabetic wounds is described here. More specifically, the present invention relates to the preparation of biocompatible Polyacrylonitrile (PAN) nanofiber scaffold comprising of Quercetin, Zinc oxide and polyacrylonitrile by electrospinning. The nanofibers formed has the average diameter of approximately 160 nm and show excellent antibacterial and wound healing potentials.

### BACKGROUND OF THE INVENTION

### 1. Field of the Invention

The present invention relates to the preparation of polyacrylonitrile nanofibers incorporated Zinc Oxide-Quercetin nanoparticles wound dressing material with antibacterial and wound healing potentials.

### 2. Description of Related Art

Chronic wounds are medical care concern and severe clinical challenge worldwide. Chronic wounds affected patients are burns victims or may be caused by skin ulcers due to venous stasis, pressure or diabetes mellitus. Diabetic foot ulcers or wounds are prevalent in patient suffering from diabetes mellitus. Wound healing failures observed in diabetic patients are caused by multiple factors including neuropathy, re-current inflammation due to high blood sugar levels that eventually leads to amputation. Conventional wound dressings like gauzes, bandages and superabsorbent cotton limits to the basic protection and do not achieve the requirement of wound care and aesthetic of the natural skin. However, multifunctional wound patches are designed to cure the skin functionalities. To accelerate the regeneration of wound tissues, dressing with swelling capacity to absorb the exudate, stretchable, enhance angiogenesis, maintains asepsis, and biocompatible nature are desired. They ensures the suitable biological environment and efficient delivery of drug to eliminate the infections without any adverse effect. Recently, non-woven electro-spun fibers have proven as excellent materials to fasten the healing process and prevent bacterial infections.

Nanofibers fabricated through electrospinning technique possess high porosity, high surface to volume ratio and creates a biomimetic system that serves as extra-cellular matrix (ECM). These characteristics of nanofibers provide suitable wound dressing scaffold that promotes skin regeneration, retains moisture, hemostasis, and cell respiration. Moreover, electrospun nanofibrous dressings are promising matrix for patient compliance as the need of frequently changing bandages is reduced. Incorporation of antimicrobial or therapeutic agents to functionalized nanofiber materials serve as potential bandages to relieve pain and promote healing that will contour to virtually any wound surface.

To fabricate nanofibrous material, polyacrylonitrile (PAN) is employed due to its biocompatibility along with excellent mechanical and thermal strength, non-toxicity, commercial availability and better spinnability. PAN nanofibers have controlled diameter ranging from 100-300 nm. In addition, zinc oxide nanoparticles owing to its antimicrobial, fibroblast proliferation and angiogenesis properties, is incorporated into PAN polymeric nanofibrous mat as an active ingredient for the wound patches.

Quercetin is a flavonoid and pharmacological active agent that delays oxidative stress and prevent cell death. It is incorporated into nanofibrous scaffold to promote the wound healing, as it possesses high antibacterial and antioxidant properties. Therefore, considering the aforementioned factors, biocompatible polymer incorporated with metal oxide nanoparticle along with nutraceutical (Quercetin) by electrospinning are invented that are suitable for the biological environment of wound to mediates healing and reduce pain.

### SUMMARY OF THE INVENTION

The present embodiment relates to the synthesis of nanofibers comprising of polyacrylonitrile, metal oxide nanoparticles loaded with nutraceuticals and its application for wound healing. The nanofibrous scaffold designed herein are loaded with Quercetin as the potential candidates. Moreover, zinc oxide has been incorporated due to the angiogenetic and anti-microbial potential. The nanofibers in the embodiment are referred to as PAN/ZnO/Quercetin (PAN/ZnO/Que). The nanofibers mat may have a diameter of 160 nm. Nanofibers in accordance with the embodiment were formed through electrospinning technique. The voltage of electrospinning was kept in the range of 10-15kV. The non-woven matrix has been designed for use as a wound dressing to cure diabetic wounds. The scaffold herein has the multifunctional properties such as biocompatibility, antimicrobial, angiogenic and drug delivery potential at the site of action. Thus, the developed dressing has the capability to cure wounds, applied over the wound to accelerate the healing, and improves the patient compliance.

### BRIEF DESCRIPTION OF DRAWINGS

FIG. 1 Depicts the electron scanning microscopy of a nanofiber comprising of PAN/ZnO/Que produced by electrospinning technique

FIG. 2 Represents the histogram showing diameter of PAN/ZnO/Que.

FIG. 3 Represents the EDX spectra of PAN/ZnO/Que.

FIG. 4 Shows the XRD plot of nanofibers scaffold PAN/ZnO/Que.

FIG. 5 Represents the FTIR spectrum of the PAN/ZnO and PAN/ZnO loaded Que.

FIG. 6 Represents the Macroscopic Visualization of wound contraction at different time points of PAN/ZnO/Que compared to the normal control.

FIG. 7 Shows the graphical representation of Wound Contraction at different time points of PAN/ZnO/Que compared to the normal control.

FIG. 8 Shows the graphical representation of Cytotoxicity of PAN/ZnO/Que compared to the Control.

FIG. 9 The graphical representation of Antibacterial Activity of PAN/ZnO/Que compared to the Control.

### DETAILED DESCRIPTION OF THE INVENTION

The current invention described herein is related to the synthesis of polyacrylonitrile based nanofibers loaded with ZnO Quercetin nanoparticles (PAN/ZnO-Que nanofibers) and their application as a biological wound dressing. PAN/ZnO-Que nanofibers according to the embodiment disclosure provides several benefits over the conventional wound patches including: biocompatibility, low cost, durability, better mechanical properties, angiogenic nature, anti-microbial potential and drug release at the target site. The polymer used in this invention is biocompatible and cheap which is impregnated with ZnO and Quercetin to enhance its additive properties in nano range. In the embodiment, the nanofibers have been produced using electrospinning.

Electrospinning is a technique to develop electrostatic fiber from electrospinnable polymer ranging from 10 nm to 1000 nm in diameter. The electrospinning instrument includes typically high voltage supply. The nanomaterial engineered through electrospinning facilitates a higher surface area to volume ratio, which mimics ECM and enables efficient encapsulation of therapeutically active agents which accelerates the wound healing. The nanofibrous scaffold designed herein are in the range of 120 nm to 200 nm dimeter. The nanofibers were processed between 10-15kV and found to be smooth.

The electrospun nanofibrous scaffold has potential attributes for wound healing. The incorporation of zinc oxide nanoparticle impart antibacterial potential to the scaffold. Moreover, Quercetin loading has equipped the scaffold with antioxidant, antibacterial and anti-inflammatory properties that promotes the healing and improves the wound contraction.

The above mentioned, factors can contribute to the design of non-woven nanofibrous dressing as competent and potential wound patch to treat chronic wounds specifically diabetic wounds that undergo amputation if treatment is delayed. The particular invention has the capability to cure diabetic wound as well as recover scar formation on the skin.

Fabrication of scaffold: To prepare the spinning solution, PAN (10% w/w) was dissolved in DMF and ZnO (0.5 wt %) and Quercetin (0.5 wt %) was loaded and stirred for 24h at 500 rpm. The solution was poured in 10 mL syringe fitted with positive Cu electrode connected with a capillary tip. The distance between the capillary tip and collector was 12 cm and the applied voltage was 11.2 kV and a flow rate of 1.3 mL/h. The nanofibers ejected from the capillary tip were collected on aluminum foil wrapped at negatively charged collector.

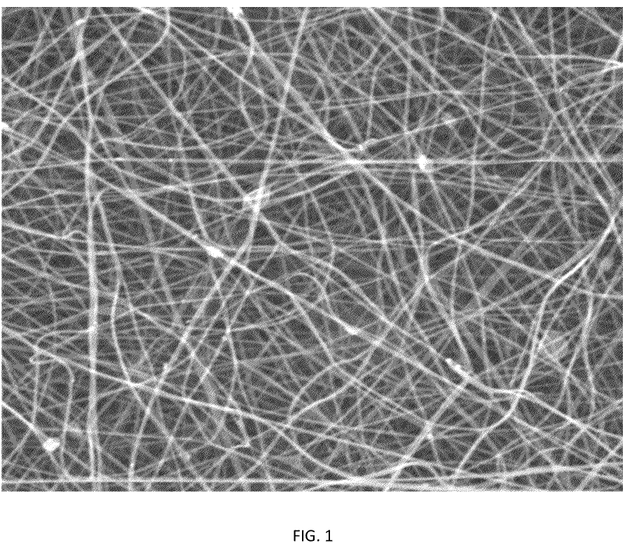

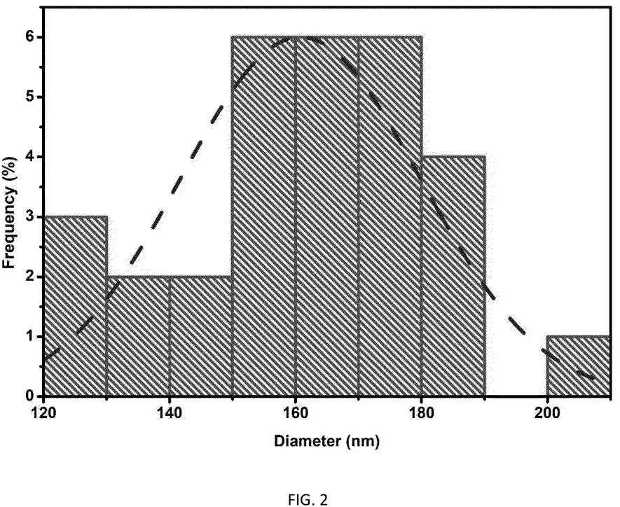

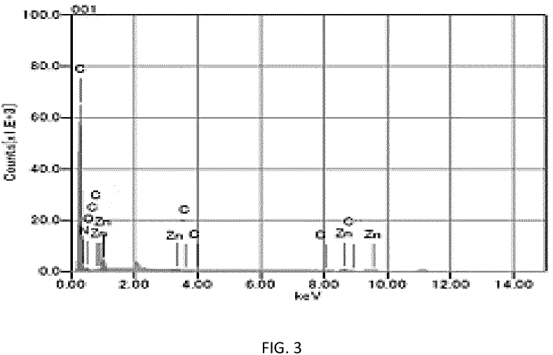

The electrospun nanofibers were analyzed for their surface morphology by scanning electron microscopy (SEM) (Model; JSM-5300, Japan). Diameter of prepared nanofibers were calculated through image J software by taking random measurements. FIG. 1 shows the surface morphology of electrospun nanofibers that represent the nanofibers are smooth but at certain position surface gets rough which may be due to agglomeration behavior of ZnO nanoparticles. The diameter was evaluated and it was found to be 160 ± 19.04 nm (FIG. 2). To confirm the presence of Zn in nanofibrous scaffold EDX analysis was performed that showed the presence of Zn, N, C and O elements as shown in FIG. 3.

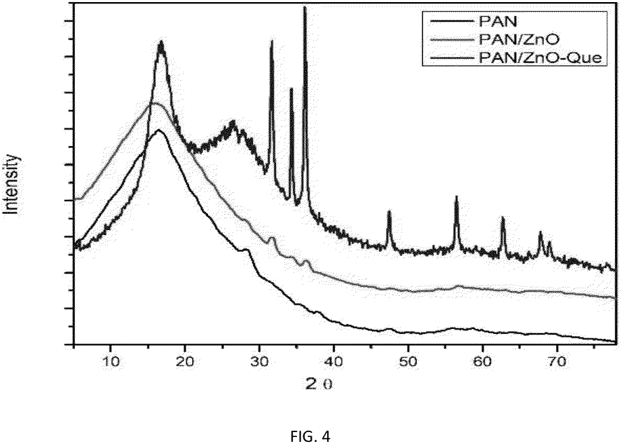

The XRD analysis of prepared nanofibers was performed at ambient temperature with sample of nanofiber on a Rotaflex RT300 mA (Rigaku manufacturer, Osaka, Japan) and nickel-filtered Cu. Ka radiation was used for measurements, along with an angular angle of 5 ≤ 2θ ≤ 80°. The XRD pattern of pristine PAN nanofiber, PAN/ZnO nanofiber and PAN/ZnO/Que nanofibers are illustrated in FIG. 4. In the pristine nanofiber a crystalline peak at 16.4° represents the PAN polymer phase. In PAN incorporated ZnO nanofibers, crystalline peak appears at 16.3° which corresponds to PAN and 32.18°, 33.9° and 36.75° implying the ZnO peaks. PAN/ZnO loaded with Quercetin shows the characteristic peaks at 27.8°, 28.3°, 29.37° along with the crystalline peak of PAN/ZnO.

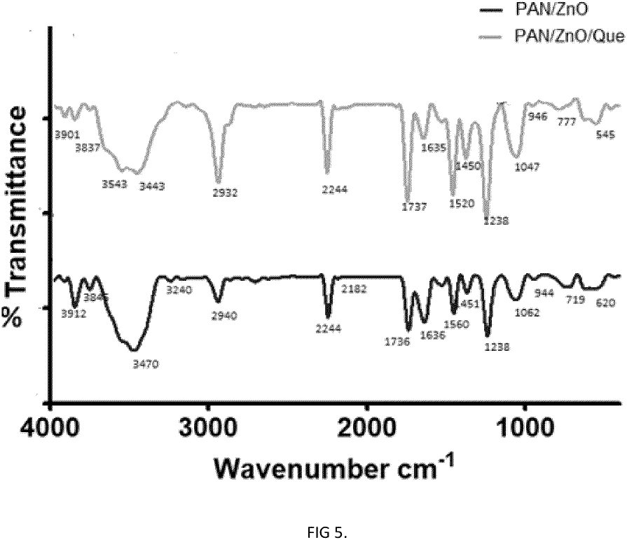

The FTIR analysis was performed to understand the possible structure of prepared nanofibrous scaffold as illustrated in FIG. 5. In both spectra the peak appearing in the region of 3400 cm-1 to 3550 cm-1 corresponds to —OH. The peaks at 2932 cm-1, 2940 cm-1, and 1450 cm-1 are due to the —CH vibration and stretching. The peaks at 1737 cm-1 and 224.80 attributes due to the stretching vibration of —C═O and —C═N respectively. In all spectra, peaks appeared at 544 cm-1 corresponds to ZnO. In PAN/ZnO/Que the aryl —C═O stretch was observed at 1635 cm-1. Aromatic —C═C ring stretch bands was evident at 1520 cm-1 and peaks at 1238 cm-1, 1047 cm-1 were attributed to stretching of C—O in phenols and —C—O—C stretch and bending in ketones.

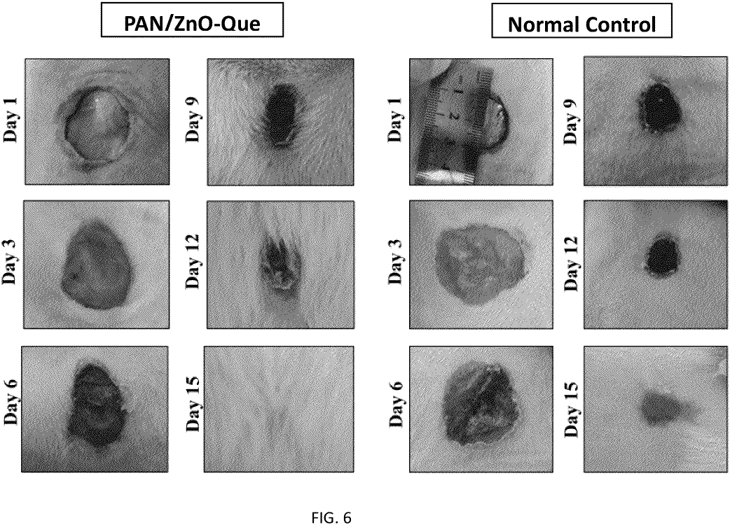

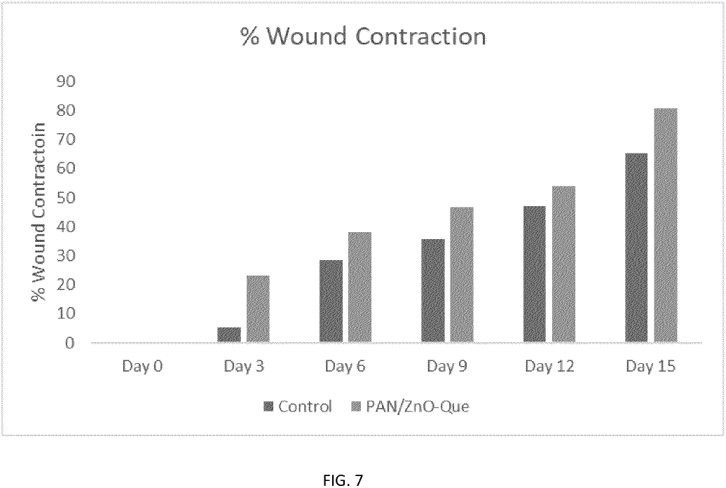

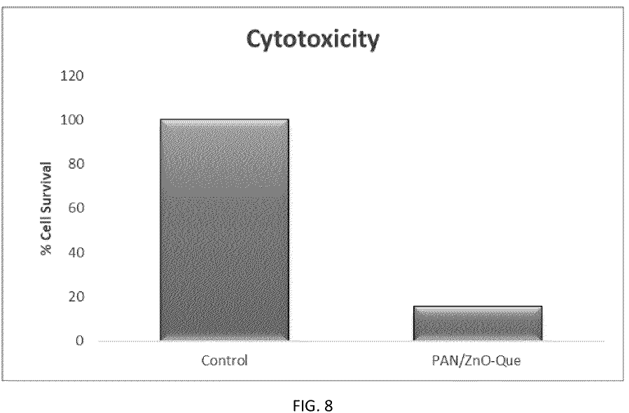

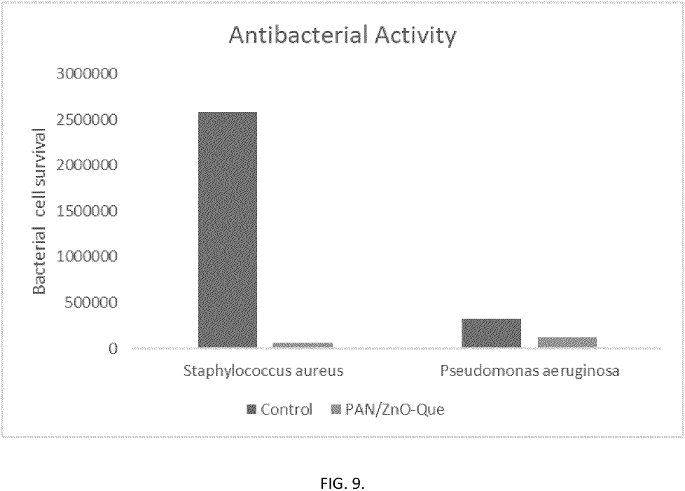

Further, in vitro, and in-vivo studies were performed to evaluate the wound healing potential of the PAN/ZnO/Que nanofibers. The % wound contraction of excision wound was measured at different time points after treatment with the nanofiber and it was observed that at day 15 the wound was completely healed compared to the control as illustrated in FIG. 6. The graphical representation of the wound contraction is also illustrated in FIG. 7, which showed 80% wound contraction at day 15 compared to the control which showed 65% contraction. [0027] Cytotoxicity was performed to evaluate the cellular toxicity of the nanofibers. The results showed only 15% cytotoxic effect of PAN/ZnO/Que compared to the control as shown in FIG. 8. The antibacterial activity was performed against Staphylococcus aureus and Pseudomonas aeruginosa which showed more than 90% bacterial inhibition compared to the control as shown in the in FIG. 9.

1. A method comprising of an electrospun matrix containing biocompatible polymer, metal-oxide nanoparticle and anti-inflammatory and anti-oxidant bioactive flavanol Quercetin.

2. The method according to claim 1, comprising 10 wt% polyacrylonitrile incorporated with 0.5 wt% ZnO in N,N-Dimethyl formamide loaded with Quercetin (0.5 wt%).

3. The prepared electrospun non-woven PAN/ZnO/Querecetin as potential scaffold for biomedical application such as wound dressings.

4. A dressing that is biocompatible with potent wound healing and antibacterial activity at the target site.

5. The method according to claim 4, for treating diabetic wounds in diabetic subjects.
